(19) United States
(12) Patent Application Publication
(10) Pub. No.: US 2003/0157164 A1
(43) Pub. Date: Aug. 21, 2003

(54) METHOD FOR THE PRODUCTION OF TABLETS BY PRESSING AND TABLETS PRODUCED BY THE METHOD

(76) Inventor: LENNART CEDGARD, GOTEBORG (SE)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 09/465,667

(22) Filed: Dec. 17, 1999

Related U.S. Application Data
(63) Continuation of application No. 09/029,336, filed on Feb. 24, 1998, now abandoned.
Application No. 09/029,336 is a 371 of international application No. PCT/SE96/01043, filed on Aug. 23, 1996.

(30) Foreign Application Priority Data
Aug. 25, 1995 (SE) 9502941-9

(51) Int. Cl. A61K 9/20; A61K 31/715; C12N 1/20; A61K 35/74

(52) U.S. Cl. 424/465; 424/93.45; 514/54; 435/252.9

(57) ABSTRACT

The invention relates to a method for the production of tablets by pressing of tablet material which comprises microorganisms. The method is characterized in that the tablet material also contains oligosaccharides, preferably inulin. The invention also includes tablets produced by the method according to the invention.

### TECHNICAL FIELD

The present invention relates to a method for the production of tablets by pressing of tablet material which contains microorganisms.

### PRIOR ART

Tablets are usually produced by pressing of a pulverulent tablet mass in a suitable shape in a so-called tablet punching machine. The tablets may have different shape and be of different size and they may also be of different hardness dependent on the properties of the tablet mass and the pressure to which they are subjected during the punching of the tablets.

When the tablets are formed heat is developed as a result of the friction against the mould surfaces and the inner friction in the tablet mass. Since the tablets usually consist of chemicals and the temperature increase is not too high, this will not create any problem since the chemicals can resist this heat increase and also are cooled rapidly. However, some tablet masses contain living microorganisms, such as bacteria, which are sensitive to high temperatures and because of this some of these bacteria die during the tablet punching.

### TECHNICAL PROBLEM

Tablets which contain microorganisms, for instance in the form of bacteria, and which are intended to contain such organisms will lose a part of or all of their value when the microorganisms are destroyed during the tablet punching. This cannot be avoided by simply using a lower pressure on the conventional tablet mass and thereby creating a lower heat development since the tablet must be subjected to a certain pressure so that it maintains its shape and is not crumbled. For known tablet masses it is not unusual that a reduction of the viability (survival) of the bacteria in the tablet is up to 80% and even more.

### SOLUTION

It has therefore always been a problem to be able to produce tablets which contain microorganisms in the form of bacteria with a lesser reduction of the viability from tablet mass to a complete tablet and therefore according to the invention a method has been obtained for the production of tablets by pressing of tablet material comprising living organisms, which is characterized in that the tablet material also contains oligosaccharides consisting of more than two monosaccharides.

According to the invention, it is suitable that the oligosaccharides consist of fructose oligosaccharides, preferably inulin.

According to the invention it is suitable that the oligosaccharides are present in an amount of 40-99.5% by weight of the tablet material.

The tablet material according to the invention can suitably contain microorganisms consisting of lactic acid producing bacteria.

The invention also comprises tablets produced by the method according to the invention, which tablets contain oligosaccharides and microorganisms whereby the oligosaccharides suitably consist of fructose oligosaccharides, preferably inulin.

The tablets according to the invention may contain lactic acid producing bacteria as microorganisms and they may also contain other additives such as polysaccharides, for example microcrystalline cellulose and starch, as well as other additives such as calcium diphosphate.

### DETAILED DESCRIPTION

The tablets according to the invention comprise microorganisms, preferably lactic acid producing bacteria cultures known as probiotica, which are intended to normalise or balance bacterial flora being present in the stomach and the intestine of humans or animals, but they may also contain other types of bacteria.

By mixing oligosaccharides, preferably fructose oligosaccharides, in the tablet mass as a so-called supporting substance the tablet punching is facilitated, which makes it possible to punch tablets at a lower pressure and lower heat development at the same time as the hardness of the tablet is maintained. The brittleness of the tablet, the friability, is surprisingly not changed with the tablet mass according to the present invention.

Due to this new composition, the punching pressure for the tablet making maybe reduced by up to 50% compared to conventional tablet punching methods without any reduction of the friability. This friability according to the invention will be 0.3-0.5, which is to be compared with the reference values which are accepted according to GMP (Good Manufacturing Practice) which are within the range of 0.1-1.0. The friability is expressed in percent weight reduction of the tablets when they are rotated 100 revolutions in a standard testing machine.

The amount of oligosaccharides depends on different crystalline qualities but may suitably be 99.5-40 weight percent of the total tablet mass without admixing any other supporting substance. However, if desired, known supporting substances such as calcium diphosphate, microcrystalline cellulose and starch may be added in a suitable small amount. A smaller addition of oligosaccharides can, however, give rise to a smaller difference with regard to the viability compared with tablet masses containing only conventional supporting substances.

The tablets according to the present invention have a lower hardness due to the lower punching pressure when the tablets are formed but an increased viability for the strain of bacteria, which makes every tablet more efficient than conventional tablets. By not pressing the tablets so hard the yield of tablets for a given amount of tablet mass will also increase.

The invention will be described more in detail below by means of two examples, of which Example 1 describes a method according to the present invention and Example 2 describes a method of conventional kind.

### Example 1: recipe having an active substance and tablet filling material

| | |
|---|---|
| Str. thermophilus & L. bulgaricus | 50% |
| Bifidobacterium animalis | 0.5% |
| L. plantaris | 0.5% |
| Inulin (fructose oligosaccharides) | 49% |
| | 100% |
| Hardness: | 2.75 kp |
| Friability: | 0.3 |
| Viability original granulate: | 5E8 cfu/g |
| Viability tablet: | 3E8 cfu/g |

40% reduction of cfu (colony forming units)

### Example 2 recipe having active substance and tablet filling material

| | |
|---|---|
| Str. thermophilus & L. bulgaricus | 50% |
| Bifidobacterium animalis | 0.5% |
| L. plantaris | 0.5% |
| Calcium diphosphate | 20% |
| Microcrystalline cellulose | 18% |
| Starch | 11% |
| | 100% |
| Hardness: | 5.5 kp |
| Friability: | 0.3% |
| Viability original granulate: | 5E8 cfu/g |
| Viability tablet: | 1E8 cfu/g |

80% reduction of cfu (colony forming units)

As appears from the above examples, the friability is maintained unchanged with a value of 0.3 whereas the hardness has been decreased to 2.75 kp compared with 5.5 kp for the conventional method. The viability has increased from 1E8 cfu/g to 3E8 cfu/g according to the invention. The reduction of cfu from tablet mass to tablet during the tablet punching became only 40% according to the new method and 80% according to the conventional method.

Accordingly, the new method results in an increased maintained viability after tablet punching of up to 200% compared with conventional tablet fillers. The increased yield results in an appreciably better economy and quality improvement of the above products.

The invention is not limited to the embodiments shown above but can be verified in different ways within the scope of the claims.

1. Method for the production of tablets having high viability in the tablet by pressing tablet material containing living microorganisms, characterized in that the tablet material also contains oligosaccharides.

2. Method according to claim 1 characterized in that the oligosaccharides are present in an amount of 40-99.5 percent by weight of the tablet material.

3. Method according to any of claims 1-2, characterized in that he oligosaccharides consist of fructose oligosaccharides.

4. Method according to any of claims 1-3, characterized in that the oligosaccharides consist of inulin.

5. Method according t any of claims 1-4, characterized in that the microorganisms consist of lactic acid producing bacteria.

6. Tablets produced according to any of claims 1-5 containing oligosaccharides and microorganisms.

7. Table according to claim 6,characterized in that the oligosaccharides consist of fructose oligosaccharides.

8. Tablets according to any of claims 6-7, characterized in that the oligosaccharides consist of inulin.

9. Tablets according to any of claim 6-8, characterized in that the microorganisms consist of lactic acid producing bacteria.

10. Tablets accorording to any of claims 6-9, characterized in that they also contain polysaccharides such as microcrystalline cellulose and starch as well as other additives such as calcium diphosphate.
